(19) United States
(12) Patent Application Publication
(10) Pub. No.: US 2007/0125662 A1
(43) Pub. Date: Jun. 7, 2007

(54) Device for treating a biological tissue volume by localise hyperthermy

(75) Inventors: Erik Dumont, Bordeaux (FR); Bruno Quesson, Bordeaux (FR)

Correspondence Address: Eric S Hyman;Blakely Sokoloff Taylor & Zafman, Seventh Floor, 12400 Wilshire Boulevard, Los Angeles, CA, 90025, US

(73) Assignee: IMAGE GUIDED THERAPY SCIENTIFIQUE UNITEC, Pessac (FR)

(21) Appl. No.: 10/584,688

(22) PCT Filed: Dec. 28, 2004
(86) PCT No.: PCT/FR04/03395

§ 371 Date: Jun. 26, 2006

(30) Foreign Application Priority Data
Dec. 30, 2003 (FR) 0315556

(51) Int. Cl. C02F 1/461; C12N 13/00

(52) U.S. Cl. 205/701; 435/173.1

(57) ABSTRACT

The inventive device for treating a biological tissue volume by a localised hyperthermy comprises a plurality of active percutaneous electrodes (1-N), at least one return electrode (120) and a high-frequency (100) electric generator for applying an alternating voltage between said active electrodes and return electrode. Said invention is characterised in that said generator powers each active electrode independently from the others in such a way that the parameters of the voltage applied by each active electrode are independently adjustable

The invention relates to a device for treating biological tissues by localized hyperthermia.

More precisely, the invention relates to a device for treating a tumor in order to degrade it by applying radiofrequency waves.

Malign tumors are generally treated by surgery (resection), by administering noxious chemical agents either globally (chemotherapy) and/or locally (e.g. by injecting ethanol), or indeed by destroying the tumor with the help of physical means. Destruction with the help of physical means consists in subjecting a cancerous zone to radiation (radiotherapy) or to heating (thermotherapy) for the purpose of irreversibly degrading the metabolism of cancer cells.

Therapy techniques involving localized hyperthermia present numerous advantages. In particular they are less traumatic for the patient and they appear to present effectiveness that is comparable to that of surgery.

These techniques consist in causing temperature to rise over a duration of a few minutes (typically from 20° C. to 40° C. for 10 minutes (min) to 20 min)) in the zone for treatment, this temperature rise being sufficient to induce necrosis by coagulation (immediate cell death) and/or by apoptosis (delayed cell death).

It is commonly accepted by practitioners specialized in treating tumors (surgeons, radiologists, radiation oncologists, oncologists) that a safety margin of 1 centimeter (cm) is needed around the volume containing the tumor in order to obtain reliable elimination of the tumor and reduce the risk of recurrence.

The reference technique for percutaneous treatment of tumors of the liver having a diameter not exceeding 3 cm is radiofrequency (RF) ablation. At present, this is the only technique that can be used as an alternative to surgery and that enables cells to be destroyed effectively over such a large volume of tissue, while ensuring that treatment durations remain reasonable for the patient (typically a few tens of minutes).

Such localized hyperthermia techniques are generally preferred to techniques of injecting noxious chemical agents locally since they enable lesions to be obtained having shapes and dimensions that are more reproducible.

Localized hyperthermia by RF is generally implemented by applying an alternating voltage between an electrode implanted in the tissue in the proximity of the target region and an outer return electrode in the form of a large-area dissipative plate positioned on the skin. The currents produced in the tissue lead to a rise in temperature that is lethal for the cancer cells located in the proximity of the electrode implanted in the tissue.

The main limitation on the effectiveness of the treatment is due to the maximum volume which it is possible to treat. Various technical solutions have been proposed for increasing this volume:
The electrode can be cooled (as proposed in particular in patent documents WO 02/056782 and U.S. Pat. No. 6,059,780). That technique makes it possible to cool the surface of the electrode implanted in the tissue and thus avoids drying out tissue that is in immediate contact with the electrode. Drying out induces a large increase in the impedance of the tissue, which reduces the magnitude of the current flow. As a result the amount of energy delivered to the tissue is much smaller and the effectiveness of the treatment is affected. The use of needle cooling thus makes it possible to avoid this drying-out effect and encourages energy delivery.
Increasing the electrical conductivity of the tissues by injecting electrically conductive substances (as proposed in particular in patent document EP 0 714 635). That technique makes it possible to maintain excellent electrical conductivity for the treated tissue and to lengthen the duration and the amount of energy that is delivered. The temperature rise is thus spread out further in three dimensions, thereby enabling the volume that can be treated from a single electrode to be increased.
The use of deployable needles of large size (as proposed in particular in patent documents U.S. Pat. No. 5,951,547, U.S. Pat. No. 6,059,780, U.S. Pat. No. 5,827,276, WO 02/22032, or WO 98/52480). Those treatment devices have a needle presenting contact area with the tissue that is increased by deploying one or more lateral elements like the ribs of an umbrella. An advantage of such “deployable needles” is that they require only one incision for putting the active elements into place. However, a drawback of such needles is that it is generally necessary to have a large number of active elements in order to obtain uniform ablation over a large volume. Local temperature distribution is associated with the number and the geometrical disposition of the active elements of the needle, and also on the potential difference between the needle and the return electrode. If the elements are too far apart or too few, ablation can be incomplete and/or the size of the lesion can be insufficient to ensure effective treatment. Using a large number of elements increases the risk of tearing and/or puncturing tissue, in particular in sensitive regions (such as, for example, the gall bladder, the hepatic dome, or the intestines).

Another drawback of such devices is that they give rise to lesions of a shape that is generally spherical or ellipsoidal, and it is very difficult to adjust the shape of the lesion to the shape of the target for treatment. Consequently, those needles are not always adapted to destroying certain tumors that are not spherical or that are located close to sensitive regions. Document US 2002/0072742 (published on Jun. 13, 2002) discloses a needle in which the elements can be deployed or retracted independently of one another in order to adapt to the shape of the volume of tissue for treatment. A radiofrequency (RF) generator feeds a rotary element that distributes electricity in succession to each element of the needle in cyclical manner. The effectiveness of treatment is not optimized since each element is activated sequentially.
The use of a bipolar needle having two electrodes, as described in document WO 02/056782. The advantage of such a device is to have two active electrodes quite close to each other, thus making it possible to concentrate electrical current between the two electrodes and to reduce the electrical power needed to induce a current of sufficient magnitude to produce a lethal temperature rise.

Nevertheless, those various approaches do not enable the shape and the dimensions of the lesion that is created to be modulated, and in order to obtain complete destruction of the tumor, it is sometimes necessary to reposition the needles in order to perform additional ablation partially overlapping the ablation of the first impact. Another problem raised by devices for hyperthermia treatment in general is that the electrical characteristics of the tissue have an influence on the current induced by the electrodes and thus on the temperature rise produced for a given potential difference.

An object of the invention is to provide a device for performing hyperthermia-treatment that is localized in a three-dimensional volume and that is adapted to treating tumors of various outlines.

Another object of the invention is to provide a device enabling tumors of large volume to be treated (typically greater than 30 cubic centimeters (cm³)).

To this end, the invention provides a method of treating a volume of biological tissue by localized hyperthermia, the device including a plurality of active percutaneous electrodes, at least one return electrode, and a high frequency electricity generator suitable for applying an alternating voltage between the active electrodes and the return electrode, the device being characterized in that the generator is suitable for feeding each active electrode independently of the others, such that the parameters of the voltage applied to each active electrode can be adjusted in independent manner.

The term “percutaneous” means that the active electrodes are suitable for being inserted deep in the tissue for treatment. They therefore require tissue to be invaded when they are put into place within the tissue.

The active electrodes may be fed independently so the device can control the local distribution of electric current within the target volume, thus making it possible to adjust the shape and the dimensions of the lesion that is created.

In particular, the amplitudes and the phase positions of the voltages applied to the electrodes can be selected so as to generate currents between the active electrodes and thus, starting from a limited number of electrodes, obtain uniform coverage in the zone for treatment.

With the device of the invention, it is consequently possible to treat tumors of large volume while using a limited number of active electrodes.

In addition, selecting the amplitudes and the phase positions of the voltages applied to the active electrodes provides flexibility to the treatment. The device gives practitioners the option of delivering energy in locations within the volume that can be adjusted or modified without necessarily having recourse to multiple repositioning of electrodes, thereby limiting the amount of tissue invasion (and reducing the risk of tumor cells being disseminated).

The various parameters that have an influence on the local distribution of temperature are the following:
the thermal characteristics of the tissue being treated (heat diffusion, blood flow, perfusion); and
the local current density, which is a function of the electrical characteristics of the tissue (electrical conductivity), of the configuration of the electrodes (number and positioning in three dimensions), and also of the voltages applied between the various electrodes.

In a preferred implementation of the invention, the treatment device comprises a plurality of active electrodes disposed in a cylindrical configuration around a return electrode.

The proposed cylindrical configuration serves to diminish the impedance between the electrodes compared with the devices that are presently in use and in which the return electrode is at a distance from the target region (a large-area cutaneous electrode). Consequently the voltage(s) for application to generate sufficient current between the electrodes is/are smaller than when using conventional devices with a cutaneous electrode.

The electrical power needed is reduced, as are the risks of destroying tissue surrounding the target region or the risks of cutaneous burning on contact with the dissipative electrode.

Nevertheless, it is possible to add one or more additional return electrodes that are placed in contact with the skin outside the target region. The advantage of such a disposition is to make it possible to favor one particular direction of electric current propagation during the intervention. The central return electrode makes it possible to increase the three-dimensional current density inside the volume defined by the active electrodes (centripetal propagation) and to increases temperature selectively in the target region. In contrast, a return electrode outside the treated region encourages centrifugal propagation of the current outwards from the same volume, thereby serving to increase the volume that is treated.

The use of such an external electrode thus makes it possible to treat the zone that is peripheral to the target region, and this is a critical factor in obtaining a sufficient safety margin to ensure that treatment is effective. These two return electrodes may be connected simultaneously (simultaneous centrifugal and centripetal propagation) or in alternation.

When they are connected simultaneously, the heat power delivered is dissipated over a larger volume than if they were being connected in alternation. Simultaneous connection increases the duration of radiofrequency application for given delivered power. A compromise can be selected by the operator or the algorithm that manages signal generation, as a function of the volume of the region for treatment.

Another advantage of the cylindrical configuration (return electrode at the center of the cylinder on which the active electrodes are distributed regularly) is to restrict in simple manner the selection of amplitudes and phases. Selecting amplitude serves to control energy delivery between each active electrode and the central return electrode, whiles selecting phases serves to control energy delivery between each active electrode and its two nearest neighboring active electrodes.

The invention is adapted to implementing a method of treating a volume of biological tissue by localized hyperthermia, the method comprising the steps consisting in:
positioning a plurality of active percutaneous electrodes and at least one return electrode in the tissue to be treated; and
applying an alternating voltage between the active electrodes and the return electrode by means of a high frequency electricity generator;

the method being characterized in that for each active electrode being fed independently of the others, the method also comprises the step consisting in adjusting the parameters of the voltage applied to each active electrode.

The step consisting in adjusting the parameters of the voltage applied to each active electrode comprise determining and setting amplitudes Vᵢ and/or phases Φᵢ for the voltages that are applied to the electrodes.

In a preferred implementation of the method, the phases Φᵢ and the voltages applied to the electrodes are determined by performing steps consisting in:
defining, for two electrodes i and j, amplitude values Vᵢ and Vⱼ for the voltages that are applied respectively thereto, and also defining a potential difference Δ that is desired between the electrodes i and j; and
deducing therefrom a phase difference Φᵢⱼ between the voltages applied to the electrodes i and j in application of the following relationship:

$${Φ}_{ij} = a cos ( \frac{{V}_{i}^{2} + {V}_{j}^{2} - {Δ}^{2}}{2 {V}_{i} \cdot {V}_{j}} )$$

Other characteristics and advantages appear further from the following description which is purely illustrative and non-limiting and should be read with reference to the accompanying figures, in which:

FIG. 1 is a diagram showing a multipole treatment device in accordance with the invention;

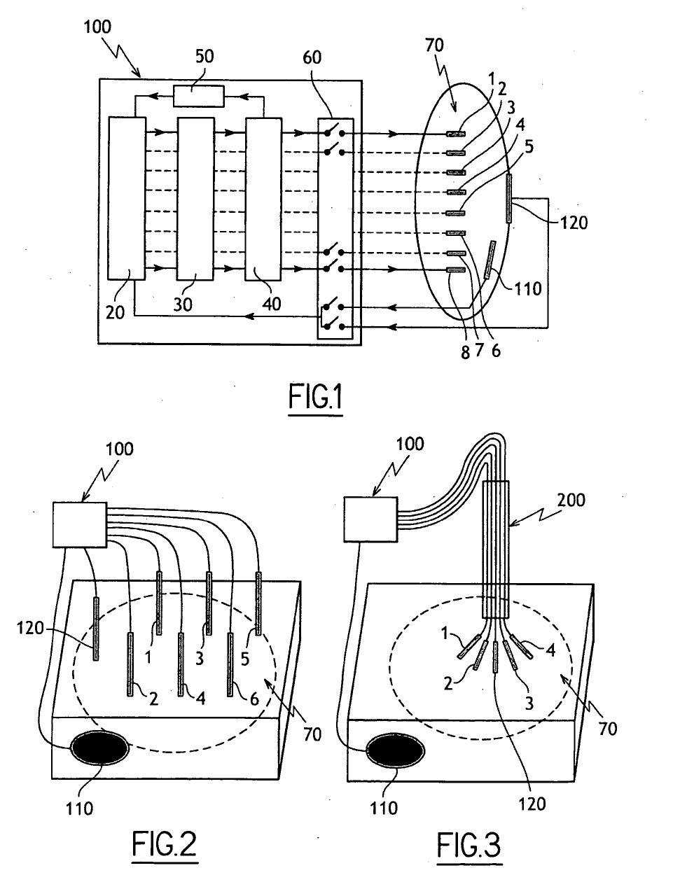

FIG. 2 is a diagram showing an implementation of the device of the invention in which the active electrodes are placed individually in the tissue for treatment;

FIG. 3 is a diagram showing an implementation of the device of the invention in which the active electrodes are deployed from a needle, thereby limiting the number of invasions needed in tissue in order to position the various electrodes;

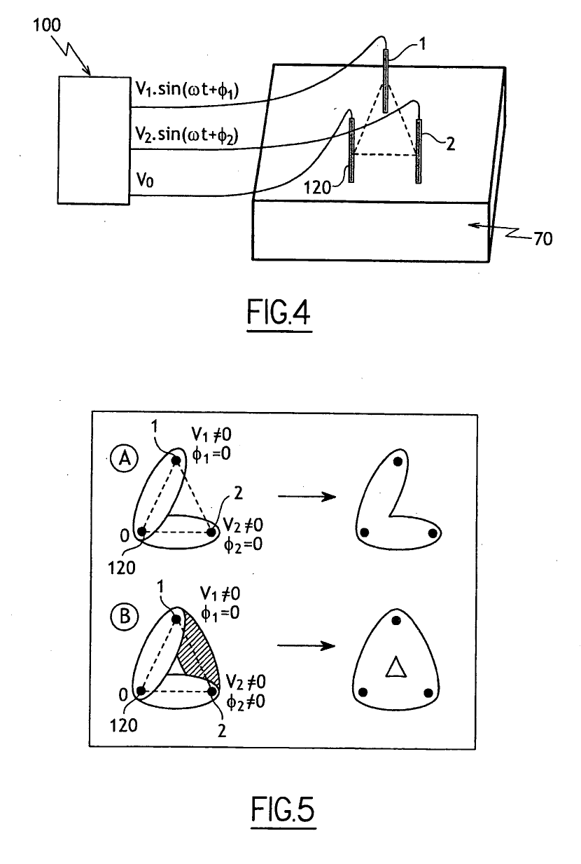

FIG. 4 is a diagram showing a device in accordance with the invention comprising two active electrodes and one return electrode;

FIG. 5 shows the two-dimensional distributions of energy delivery in the treated tissue as a function of the voltages applied to the electrodes of the FIG. 4 device;

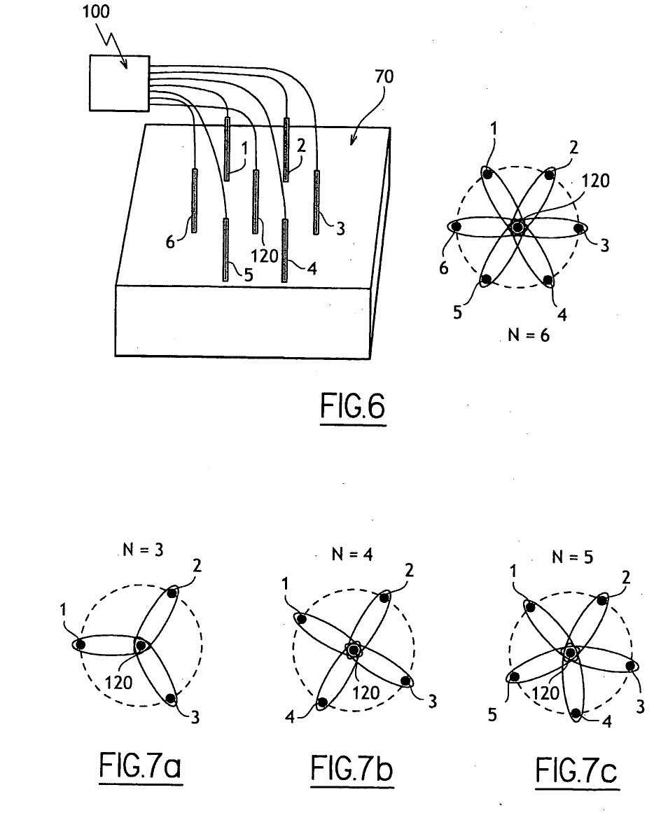

FIG. 6 is a diagram showing a configuration of electrodes that enables uniform tissue necrosis to be obtained;

FIGS. 7A, 7B, and 7C are diagrams showing the two-dimensional distributions of energy for a device respectively comprising three, four, or five active electrodes, with the voltages applied to each of the electrodes being identical in amplitude;

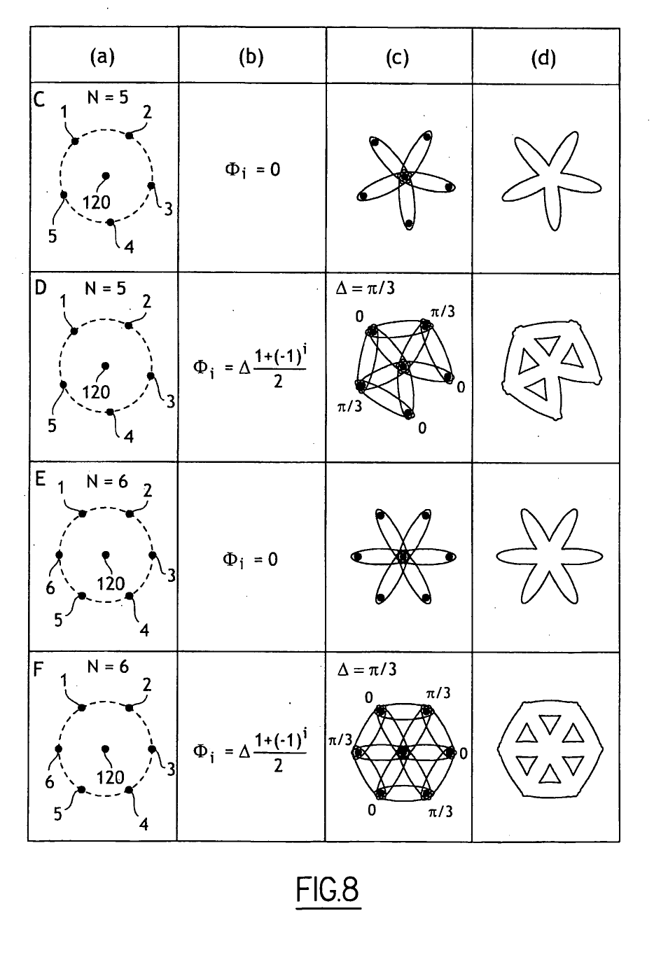

FIG. 8 is a table showing various two-dimensional distributions of energy delivery that can be obtained by applying feed voltages presenting amplitudes that are identical and by adjusting the phase differences between the electrodes; and

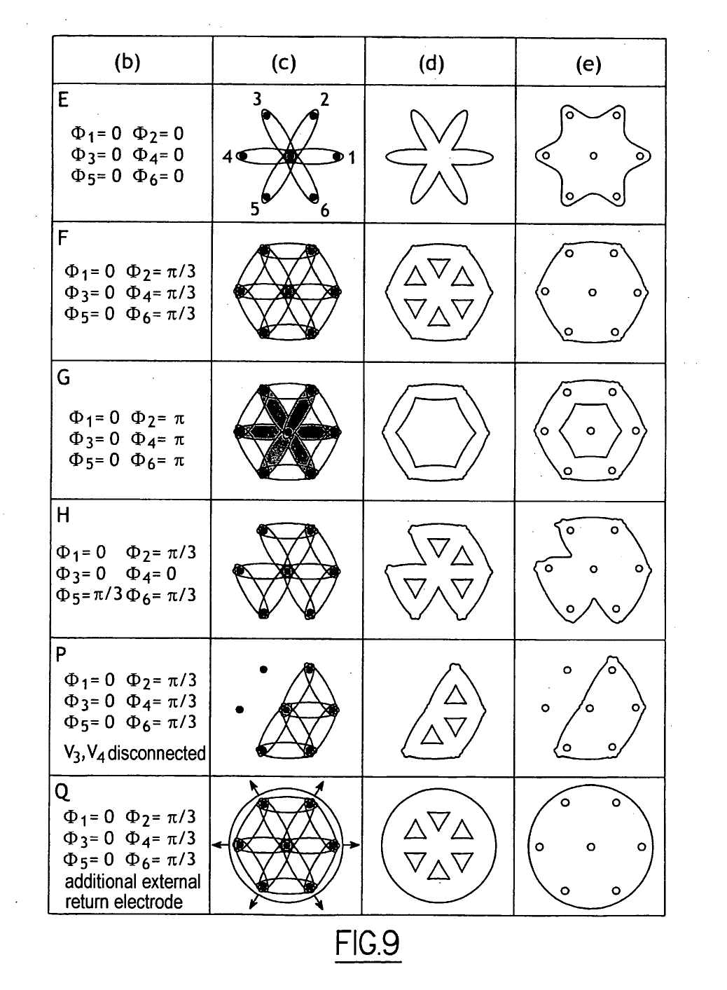

FIG. 9 is a table showing the different shapes of necrosis that can be generated with a device having six active electrodes and one return electrode, by adjusting the voltage phase differences between the electrodes and by connecting and/or disconnecting certain electrodes.

In FIG. 1, the treatment device comprises a multichannel generator 100 having multichannel means 20 for generating sinusoidal voltages that are controllable in amplitude and in phase position, together with amplification means 30 for amplifying the voltages as generated in this way. The generator also has measurement means 40 for measuring the electrical characteristics of each channel (delivered current and voltage), and control means 50 responsive to the measured electrical characteristics to control the voltage generator means 20 to adjust the power delivered by each channel.

The treatment device further comprises a plurality of active transcutaneous electrode 1 to 8 implanted in a target zone 70 of biological tissue for treatment, and transcutaneous return electrodes 110 and 120 likewise implanted close to the target zone 70. Each active electrode 1 to 8 is connected to one of the channels of the multichannel generator 100 and is fed with voltage independently of the other electrodes. The return electrodes 110 and 120 are connected to the reference channel (floating ground) of the generator 100.

A set of switches 60 serves to connect or disconnect each of the electrodes 1 to 8, 110, and 120 independently of one another. The switches may be controlled manually and/or automatically (e.g. by a system of electro-mechanical relays).

The FIG. 1 treatment device constitutes a multipole treatment device insofar as the electrodes are controlled simultaneously and independently of one another.

FIGS. 2 and 3 are diagrams showing two possible implementations of the invention.

In the implementation shown in FIG. 2, the active electrodes 1 to 8 and one of the return electrodes 120 are implanted separately in the volume 70 of tissue for treatment. Each electrode requires an incision in order to be implanted, and the electrodes may be disposed relative to one another in a multitude of configurations. In this figure, the other return electrode is in the form of a dissipative plate placed on the surface of the tissue for treatment.

In the implementation shown in FIG. 3, the active electrodes 1 to 8 and one of the return electrodes 120 are implanted by means of a needle 200 from which the electrodes are deployed. In this figure likewise, the other return electrode 110 is in the form of a dissipative plate placed on the surface of the tissue for treatment.

In FIG. 4, the treatment device comprises a multichannel generator 100 with two channels connected to two active percutaneous electrodes 1 and 2 and with the reference channel connected to a percutaneous return electrode 120. The three electrodes 1, 2, and 120 are implanted in the volume of tissue 70 for treatment in an equilateral triangle configuration.

The active electrodes 1 and 2 are fed by the generator 100 with respective voltages of amplitudes V₁ and V₂ and phases Φ₁ and Φ₂. The following thus applies:
V₁(t)=V₁·sin(ωt+Φ₁)
V₂(t)=V₂·sin(ωt+Φ₂)
V₁₂₀(t)=V₀
where V₀ is the reference potential of the return electrode 120 (generally and by convention in this example V₀=0).

FIG. 5 shows the two-dimensional distributions of energy delivery (represented by ellipses) within the treated tissue 70 when V₁=V₂ and when there is no phase difference between the active electrodes (Φ₁=Φ₂) (distribution A) or when there is a phase difference between the active electrodes (Φ₁≠Φ₂) (distribution B). This figure also shows the shapes of the necroses that are obtained for each distribution.

The device of FIG. 5 is particularly simple and inexpensive, it implements only two active electrodes 1 and 2, together with a generator feeding two channels.

FIG. 6 shows a preferred implementation of the invention in which the treatment device comprises a plurality of percutaneous active electrodes 1 to N disposed in a cylindrical configuration and spaced apart regularly, together with a percutaneous return electrode 120 disposed in the center of the cylinder.

Advantageously, the percutaneous active electrodes are six in number (N=6), such that the distance between two successive active electrodes is equal to the distance between an active electrode and the central return electrode. The use of a geometrical disposition that is symmetrical around the return electrode 120 favors obtaining a uniform distribution of temperature within the target region while using a small number of electrodes.

If it is assumed that the electrical characteristics of the tissue are uniform throughout the target region 70, it follows that the impedances between each of the electrodes 1 to N and the return electrode 120 are substantially equal.

Applying identical voltages to each of the active electrodes 1 to N generates similar currents between each active electrode and the central return electrode 120.

Another advantage of this cylindrical disposition is to diminish the impedance between the electrodes compared with systems presently in use in which the return electrode is at a distance from the target region (a large area plate). Consequently, energy delivery is confined to within the target region. The voltage(s) for application in order to generate sufficient current between the electrodes is/are therefore smaller than in the conventional configuration, thus reducing the electrical power needed, and also reducing the risk of destroying tissue surrounding the target region or of burning at the contact with the dissipative cutaneous electrode.

FIGS. 7A, 7B, and 7C are diagrams showing the two-dimensional energy distributions for a device comprising respectively N=3, 4 and 5 active electrodes disposed in a cylindrical configuration, when the amplitudes and the phases of the voltages applied to each active electrode are identical.

FIG. 8 is a table showing various two-dimensional distributions of energy delivery that can be obtained by adjusting the phase differences of the voltages between the electrodes for a device having five active electrodes (configurations C and D) and for a device having six active electrodes (configurations E and F) distributed regularly around a cylinder centered on the return electrode. In this table, column (a) gives the configuration in question, column (b) gives the phase of the voltage applied to each electrode i, column (c) gives the two-dimensional distribution of current as generated between the electrodes, and column (d) gives the resulting distribution of heating.

In configuration C, the five active electrodes are fed with voltages presenting identical amplitudes and phases. The currents generated in the tissue for treatment are localized between each active electrode and the return electrode. It follows that the two-dimensional distribution of energy delivered in the tissue has the general shape of a five-branched star centered on the return electrode, with each branch extending to one of the active electrodes.

In configuration D, the five active electrodes are fed with voltages presenting identical amplitudes. Three of the active electrodes are fed with voltages presenting a phase of zero, and the other two are fed with voltages presenting phase positions of π/3. The currents generated in the tissue for treatment are localized firstly between each active electrode and the return electrode, and secondly between successive active electrodes, with the exception of the two successive active electrodes which are fed with voltages presenting phase positions of zero. As a result, the two-dimensional distribution of energy delivered in the tissue presents overall the shape of an incomplete pentagon.

In configuration E, the six active electrodes are fed with voltages presenting identical amplitudes and phases. The currents generated in the tissue for treatment are localized between each active electrode and the return electrode. It follows that the two-dimensional distribution of energy delivered in the tissue presents the general shape of a six-branched star centered on the return electrode with each branch extending to one of the active electrodes.

In configuration F, the six active electrodes are fed with voltages presenting amplitudes that are identical. The electrodes are fed with voltages presenting, in alternation, phase positions of zero and of π/3. The currents generated in the tissue for treatment are localized firstly between each active electrode and the return electrode, and secondly between successive active electrodes. It follows that the two-dimensional distribution of energy delivered in the tissue presents the overall shape of a hexagon. This configuration favors high current density in the inter-electrode space. The even number of electrodes makes it possible to apply identical phase differences between pairs of successive active electrodes. In contrast, an odd number of electrodes makes such a configuration impossible, unless the phase difference between two successive active electrodes is equal to 2π/N (here N=5). However that determines the phase positions and it is no longer possible to modulate the maximum voltage between two consecutive active electrodes.

FIG. 9 is a table showing various two-dimensional distributions of energy delivery that can be obtained by adjusting the phase positions of the voltages between the electrodes for a device having six electrodes. The six electrodes are disposed in a cylindrical configuration centered on a return electrode, possibly together with an additional return electrode in the form of a cutaneous conductive plate. The electrodes 1 to 6 are fed with voltages presenting amplitudes that are identical. Column (b) gives the phase position of the voltage applied to each electrode i, column (c) gives the two-dimensional distribution of the current generated between the electrodes, column (d) gives the distribution of heating, and column (e) shows the shape of the resulting necrosis.

With configuration F (successive phase positions of 0 and π/3), the shape of the resulting necrosis is more circular (ideal case) than with configuration E.

This configuration can be obtained with a generator having two feed channels by connecting three active electrodes to each of the channels of the generator. Advantageously, the applied voltages can be at phase differences of π/3 relative to one another. It then suffices to connect the odd active electrodes (1, 3, and 5) to one of the channels and the even active electrodes (2, 4, and 6) to the other channel. This system is thus simpler and less expensive to implement than a system having six independent channels, even though it offers less flexibility.

With configuration G (successive phase positions of 0 and π), the voltage between successive active electrodes is twice the voltage between each active electrode and the return electrode (shaded ellipses). Consequently, energy delivery is essentially distributed on a ring containing the six active electrodes.

With configuration H (identical to configuration F but with the voltages applied to electrodes 4 and 5 being interchanged), the temperature distribution is identical to that for configuration F, except between electrodes 3 & 4 and 5 & 6 which have voltages that are identical.

With configuration P (identical to configuration F, but with electrodes 3 and 4 disconnected), the temperature distribution is identical to that of configuration F for electrodes 1, 2, 5, and 6, and is zero around electrodes 3 and 4.

With configuration Q (identical to configuration F, together with an outer dissipative plate), the temperature distribution is identical to that for configuration F, but extends outwards further from the cylinder formed by the active electrode. This configuration makes it possible to increase the outside volume of the treated region and to generate a safety margin.

On sight of FIG. 9, it can be understood that, with a given number of electrodes organized in a certain configuration, the device of the invention makes it possible to generate necroses having a multiplicity of shapes.

Depending on the shape of the tumor, and on the characteristics of the tissue, it is possible to achieve ablation by applying a sequence of successive configurations. Combining configurations makes it possible to modulate the shape of the necrosis that is generated even more precisely.

The number of active electrodes may also be modified by connecting or disconnecting certain electrodes.

In general, if each electrode i is subjected to a potential Vᵢ(t) having the form:
Vᵢⱼ(t)=Vᵢⱼ·sin(ωt+Φᵢⱼ) [1]
the potential difference between electrodes i and j is given by:
Vᵢⱼ(t)=Vᵢⱼ·sin(ωt+Φᵢⱼ), with Vᵢⱼ≧0 [2]
where Vᵢⱼ and Φᵢⱼ are respectively the amplitude and the phase of the voltage generated between electrodes i and j, with:

$$\begin{matrix}{V}_{ij} = \sqrt{{V}_{i}^{2} + {V}_{j}^{2} - 2 {V}_{i} \cdot {V}_{j} \cdot cos ( {Φ}_{i} - {Φ}_{j} )} , \\ where {V}_{ij} ∈ [ \vert {V}_{j} - {V}_{i} \vert , {V}_{i} + {V}_{j} ] & [ 3 ] \\ {Φ}_{ij} = a tan ( \frac{{V}_{j} sin {Φ}_{j} - {V}_{i} sin {Φ}_{j}}{{V}_{j} cos {Φ}_{j} - {V}_{i} cos {Φ}_{i}} ) & [ 4 ]\end{matrix}$$

When the potentials Vᵢ and Vⱼ are identical, the potential difference Vᵢⱼ can be adjusted over the range 0 to 2Vᵢ as a function of phase difference. It is thus possible to favor localized energy delivery between these two electrodes since the voltage Vᵢⱼ can be up to twice the magnitude of the voltage between each active electrode and the return electrode (Vᵢ, Vⱼ).

If the phase difference is equal to 0 (conventional devices having a single feed channel), the potential differences between all of the active electrodes are zero, regardless of the voltages Vᵢ and Vⱼ.

If the phase difference is equal to π/3, and the voltages Vᵢ and Vⱼ are identical, the potential differences between all of the electrodes are identical, thereby making energy delivery more uniform.

If the phase difference is equal to π and the voltages Vᵢ and Vⱼ are identical, then the potential differences between the active electrodes are equal to 2Vᵢ and the amount of energy delivered is greater between these electrodes than it is around the return electrode.

Equation [3] makes it possible to determine the potential difference between electrodes i and j on the basis of the amplitudes and the phases of the potentials that are applied thereto. By rewriting equation [3], it is possible to determine the phase difference that makes it possible to obtain a desired potential difference Δ between the electrodes i and j:

$${Φ}_{ij} = a cos ( \frac{{V}_{i}^{2} + {V}_{j}^{2} - {Δ}^{2}}{2 {V}_{i} \cdot {V}_{j}} ) , with Δ ∈ [ \vert {V}_{j} - {V}_{i} \vert , {V}_{i} + {V}_{j} ] \qquad [ 5 ]$$

This formula is applicable regardless of the number of electrodes, thereby making it possible to determine the phase differences that enable desired potential differences to be obtained between the various electrodes. The selected amplitude and phase for each independent electrode makes it possible to provide greater flexibility in treatment, since it gives the practitioner the option of delivering energy which can be located in two dimensions in a manner that can be adjusted without repositioning electrodes.

For a generator possessing N independent channels, it is possible to specify N amplitudes and N phases, thereby leading to 2N adjustable values. This number of adjustable parameters thus gives greater flexibility compared with generator systems that posses only one channel. It should be observed that for a system possessing N independent active electrodes and one return electrode, the total number of inter-electrode voltages is equal to N·(N+1)/2. Table 1 gives a list of the variables and the voltages as a function of the number of active electrodes. For a system having fewer than three electrodes, the system is mathematically over-dimensioned, since it has more variables than voltages.

For a system having three electrodes, the system is properly dimensioned since there are as many variables as there are voltages. However, for a system having more than three electrodes, the number of voltages is greater than the number of adjustable variables and it is therefore necessary to achieve a compromise in the selection of the electrodes to which the voltages are to be adjusted. For unchanging potential difference, the local distribution of current increases with decreasing distance between electrodes. Consequently, one solution consists in restricting available voltages for adjustment to those electrodes that are closest to a determined active electrode.

TABLE 1

| | Number of | |
|---|---|---|
| Number of active | variables | Number of voltages |
| electrodes | Vᵢ, Φᵢ | Vᵢⱼ |
| 2 | 4 | 3 |
| 3 | 6 | 6 |
| 4 | 8 | 10 |
| 5 | 10 | 15 |
| 6 | 12 | 21 |
| 7 | 14 | 28 |
| 8 | 16 | 36 |
| 9 | 18 | 45 |
| 10 | 20 | 55 |

In order to obtain identical energy delivery between the active electrodes, it is necessary to apply identical phase differences between pairs of consecutive electrodes. One solution consists in alternating the phase position between Δ and 0 in the order of electrode placing, such that:

$${Φ}_{i} = Δ \cdot \frac{( 1 + {( - 1 )}^{i} )}{2} , with i ∈ [ 1 , N ] \qquad [ 6 ]$$

If the number of active electrodes is odd (N=2·p+1, for integer p), the first and last phase positions are necessarily identical (giving a difference of 0) so alternation is not complied with. The only solution that enables identical phase differences to be obtained between successive pairs of electrodes is to impose a total phase difference of 2π over the set of all of the electrodes. Under such conditions, the phase of the i^{th} electrode is given by:

$${Φ}_{i} = \frac{2 i π}{N} \qquad [ 7 ]$$

where N is the total number of active electrodes.

This thus determines the phase difference as a function of the number of electrodes so it is no longer possible to select the voltage values between the electrodes since each phase is determined.

In contrast, if the radiofrequency needle has an even number of electrodes (N=2·p, for integer p), it is possible to set an identical phase difference between any two successive electrodes in order to obtain the desired potential difference Δ (equations [5] and [6]).

It is therefore preferable for the number of active electrodes to be even so as to ensure adjustable and identical phase differences (equation [6]) between any two successive active electrodes and thus take advantage of the multipole aspect.

Another possibility offered by applying radiofrequencies using a multipole device of the invention is to be able to disconnect one or more electrodes from the power supply while treatment is taking place. This can be achieved using switches that are manual, or that are electronically controlled via a relay system. The advantage of such a device is that it enables an electrode to be made inactive by opening the circuit connecting it to the return electrode or to one of the channels of the multichannel generator. The advantage is to avoid inducing a temperature rise in the proximity of said electrode, for example if it is located close to a “sensitive” region.

Means for monitoring local energy delivery as available on clinical appliances and relying on measuring impedance between electrodes or on taking local temperature measurements using implanted probes (thermocouples) can likewise be integrated in the device proposed by the present invention. For example, the device of the invention may include means for measuring impedance between electrodes and/or means for measuring local temperatures, together with means for controlling the voltages applied by the generator to the electrodes as a function of the impedance measurements and/or the temperature measurements performed continuously while the radiofrequency is being applied.

1. A method of treating a volume of biological tissue by localized hyperthermia, the device including a plurality of active percutaneous electrodes (1-N), at least one return electrode (120), and a high frequency electricity generator (100) suitable for applying an alternating voltage between the active electrodes (1-N) and the return electrode (120), the device being characterized in that the generator (100) is suitable for feeding each active electrode (1-N) independently of the others, such that the parameters of the voltage applied to each active electrode can be adjusted in independent manner.

2. A device according to claim 1, characterized in that the electricity generator (100) includes means (20) for adjusting the amplitude and the phase of the voltage applied to each active electrode (1-N).

3. A device according to claim 2, characterized in that the generator is suitable for applying voltages to two active electrodes i and j that present respective amplitudes Vᵢ and Vⱼ with a phase difference Φᵢⱼ between the voltages that is equal to:
$${Φ}_{ij} = a cos ( \frac{{V}_{i}^{2} + {V}_{j}^{2} - {Δ}^{2}}{2 {V}_{i} \cdot {V}_{j}} ) , Δ ∈ [ \vert {V}_{j} - {V}_{i} \vert , {V}_{i} + {V}_{j} ]$$
where Δ is a desired potential difference between the electrodes i and j, and Vᵢ is the amplitude of the potential difference between the i^{the} electrode and the return electrode.

4. A device according to any preceding claim, characterized in that the electricity generator (100) is a multichannel voltage generator.

5. A device according to any preceding claim, characterized in that the generator (100) includes a set of manually or automatically controlled switches (60), the switches being suitable for independently activating or deactivating feed to one or more electrodes.

6. A device according to any preceding claim, characterized in that it includes a plurality of active electrodes (1-N) disposed at equal distances from a percutaneous return electrode (120).

7. A device according to any preceding claim, characterized in that it has an even number of active electrodes (N=2·p, for integer p).

8. A device according to claims 6 and 7, characterized in that it has six active electrodes (1-6) distributed in uniform manner in a cylindrical configuration, the return electrode being disposed at the center of the cylinder.

9. A device according to any one of claims 6, 7, and 8, characterized in that the generator (100) is suitable for providing feed voltages presenting phase differences that alternate between consecutive pairs of electrodes.

10. A device according to claim 6 or claim 7, characterized in that the generator (100) is suitable for supplying feed voltages presenting equal phase differences between successive pairs of electrodes.

11. A device according to any preceding claim, characterized in that it includes an additional, external return electrode (11), in particular in the form of a cutaneous conductive plate.

12. A device according to any preceding claim, characterized in that it includes means for measuring impedance between electrodes and/or means for taking local temperature measurements, and means for controlling the applied voltages as a function of the impedance and/or temperature measurements taken.

13. A method of treating a volume of biological tissue by localized hyperthermia, the method comprising the steps consisting in:
positioning a plurality of active percutaneous electrodes (1-N) and at least one return electrode (120) in the tissue to be treated; and
applying an alternating voltage between the active electrodes (1-N) and the return electrode (120) by means of a high frequency electricity generator (100);
the method being characterized in that for each active electrode (1-N) being fed independently of the others, the method also comprises the step consisting in adjusting the parameters of the voltage applied to each active electrode (1-N).

14. A method according to claim 13, characterized in that the active electrodes (1-N) are disposed in a cylindrical configuration around the percutaneous return electrode (120).

15. A method according to claim 14, characterized in that six active electrodes (1-6) are distributed uniformly around a cylindrical configuration, the return electrode (120) being disposed in the center of the cylinder.

16. A method according to any one of claims 13 to 15, characterized in that the step consisting in adjusting the parameters of the voltage applied to each active electrode (1-N) includes independently activating and deactivating the feed to one or more electrodes.

17. A method according to any one of claims 13 to 16, characterized in that the step that consists in adjusting the parameters of the voltage applied to each active electrode (1-N) comprises determining and setting the amplitudes Vᵢ and/or the phases Φᵢ of the voltages applied to the electrodes.

18. A method according to claim 17, characterized in that the phases Φᵢ of the voltages applied to the electrodes (1-N) are determined in application of the steps consisting in:
defining, for two electrodes i and j, amplitude values Vᵢ and Vⱼ for the voltages that are applied respectively thereto, and also defining a potential difference Δ that is desired between the electrodes i and j; and
deducing therefrom a phase difference Φᵢⱼ between the voltages applied to the electrodes i and j in application of the following relationship:
$${Φ}_{ij} = a cos ( \frac{{V}_{i}^{2} + {V}_{j}^{2} - {Δ}^{2}}{2 {V}_{i} \cdot {V}_{j}} ) , with Δ ∈ [ \vert {V}_{j} - {V}_{i} \vert , {V}_{i} + {V}_{j} ]$$

19. A method according to claim 17, characterized in that the active electrodes (1-N) are disposed in a cylindrical configuration around the return electrode, and the generator (100) is controlled to deliver feed voltages presenting alternating phase differences between consecutive pairs of electrodes.

20. A method according to claim 17, characterized in that the active electrodes (1-N) are disposed in a cylindrical configuration around the return electrode, and the generator (100) is controlled to supply feed voltages presenting equal phase differences between successive pairs of electrodes.
